(19) United States
(12) Patent Application Publication
(10) Pub. No.: US 2012/0065472 A1
(43) Pub. Date: Mar. 15, 2012

(54) PROTECTIVE SHEATH

(76) Inventors: Mark Doyle, Del Mar, CA (US); How-Lun Chen, San Diego, CA (US)

(21) Appl. No.: 12/880,061

(22) Filed: Sep. 11, 2010

(51) Int. Cl. A61M 25/00; A61B 1/00; A61B 18/18

(52) U.S. Cl. 600/121; 604/171; 606/49

(57) ABSTRACT

A protective sheath including a first portion configured to receive a medical instrument and a second portion configured to accommodate movement of a distal end of said medical instrument within the second portion. The second portion is movably coupled to the first portion. The protective sheath is configured to control contamination of the medical instrument when the medical instrument and the protective sheath are placed within a patient. The protective sheath also includes a tube configured to assist the medical instrument when the medical instrument and the protective sheath are placed within said patient. The tube is disposed along a length of the protective sheath.

### BACKGROUND

Surgical devices, such as laparoscopic devices, entering a patient must be properly cleaned due to fouling and contamination. Cleaning of the devices can be very labor intensive.

Typically, such devices utilize electrosurgery energy that may cause harm to the patient when the devices are inside the patient. Moreover, multiple functions of such devices are often required during laparoscopic surgery.

### BRIEF DESCRIPTION OF THE DRAWINGS

FIG. 1 illustrates an example of a hand-actuated articulating surgical tool, in accordance with an embodiment of the present invention.

FIGS. 2A-B illustrate examples of a control portion, in accordance with embodiments of the present invention.

FIGS. 3A-7C and 9A-D illustrate examples of modules, in accordance with embodiments of the present invention.

FIG. 8 illustrates an example of a tool, in accordance with an embodiment of the present invention.

FIGS. 10A-12 and 14A-B illustrate examples of protective sheaths, in accordance with embodiments of the present invention.

FIG. 13 illustrates an example of a flow chart of a method for protecting a medical instrument, in accordance with an embodiment of the present invention.

FIG. 15 illustrates an example of a flow chart of a method for assisting a medical instrument, in accordance with an embodiment of the present invention.

The drawings referred to in this description should be understood as not being drawn to scale except if specifically noted.

### DESCRIPTION OF EMBODIMENTS

Reference will now be made in detail to embodiments of the present technology, examples of which are illustrated in the accompanying drawings. While the technology will be described in conjunction with various embodiment(s), it will be understood that they are not intended to limit the present technology to these embodiments. On the contrary, the present technology is intended to cover alternatives, modifications and equivalents, which may be included within the spirit and scope of the various embodiments as defined by the appended claims.

Furthermore, in the following description of embodiments, numerous specific details are set forth in order to provide a thorough understanding of the present technology. However, the present technology may be practiced without these specific details. In other instances, well known methods, procedures, components, and circuits have not been described in detail as not to unnecessarily obscure aspects of the present embodiments.

### Embodiments of Hand-Actuated Articulating Surgical Tool

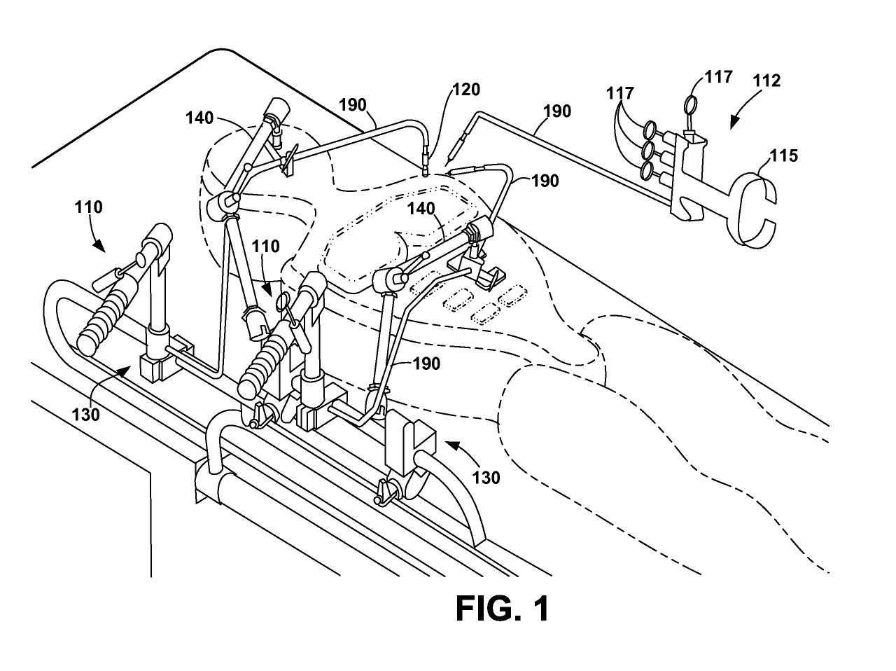

FIG. 1 shows a surgical tool according to an embodiment of the present invention. The tool has a control portion 110, 112 at the proximal end of the device and a slave portion 120 at the distal end of the device. As used herein, “proximal” refers to the part of the device that remains outside the patient's body, closest to the user. “Distal” refers to the end inserted into the patient, farthest away from the user. As with a specific component of the device, “proximal” refers to the part of the component closest to the proximal end of the device, whereas “distal” refers to the part of the component closest to the distal end of the device. An intermediate portion 190 lies between the control portion 110 and the slave portion 120. The “slave portion,” or the “distal end of the device,” 120 is the portion of the device comprising the slave modules, i.e., the extend module, the bend module, the rotate module, and the grasp module, as each is described in greater detail below. Each portion will now be described in greater detail. The term “cannula” is used to refer to the portion of the device comprising both the intermediate portion 190 and the slave portion 120.

The control portion 110, 112 may be any device that can translate the movements of the user's hand and fingers into hydraulic, mechanical, or electrical signals to actuate the corresponding parts of the slave portion 120 of the device. For example, two such devices are shown in FIG. 1.

In certain embodiments, the control portion 110, 112 uses hydraulic fluid to transfer pressure from a control cylinder to a slave cylinder. The fluid is preferably sterilized distilled water, however a saline solution, a perfluorinated hydrocarbon liquid, or any other physiologically compatible fluid could also be used. A “physiologically compatible fluid” is a fluid that once exposed to tissues and organs, does not create any intolerable reaction, such as a rash or immune response, in the patient, and does not adversely interfere with the normal physiological function of the tissues or organs to which it is exposed. In addition, a physiologically compatible fluid can remain in a patient's body or in contact with a tissue or an organ without the need to remove the fluid.

In one embodiment, the control portion 112 clamps onto the arm of the user by way of a clamp 115. The control portion 112 features finger loops 117, into which the user inserts the user's fingers. By squeezing each finger loop 117, the user creates hydraulic pressure or an electrical signal that results in a corresponding motion at the distal end 120 of the device. The user may then “open” the squeezed finger to create the opposite motion.

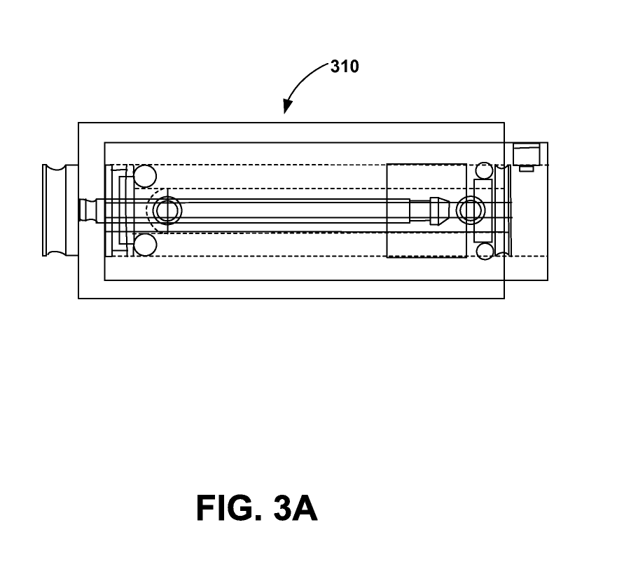

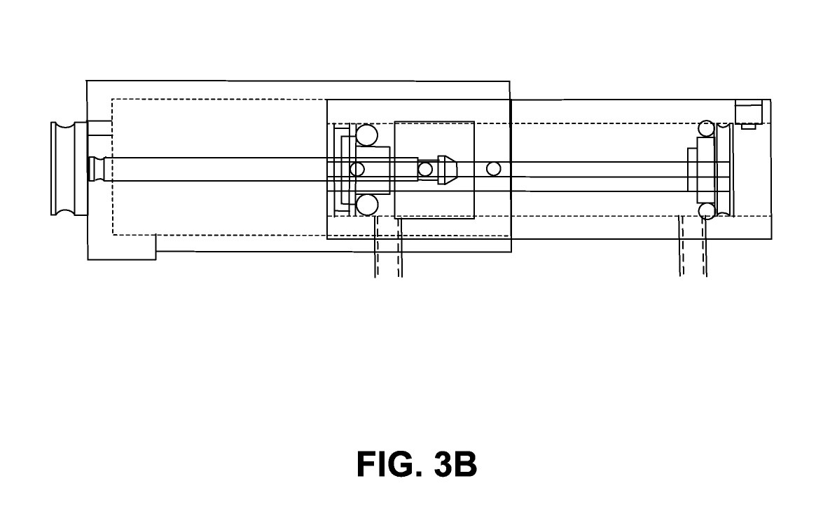

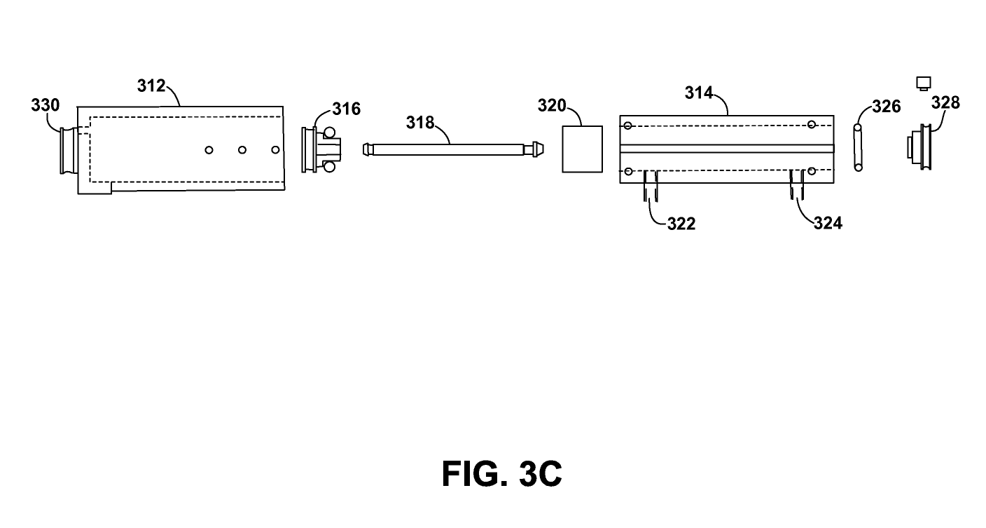

Each finger loop 117 is connected with a control cylinder 310 (shown in FIG. 3). The finger loop 117 should be large enough to allow comfortable insertion of a human finger. The finger loop 117 is connected to a longitudinal shaft. The shaft may be made of, for example, metal, ground glass, or ceramic. The shaft may be of any cross-sectional shape. In one embodiment, the cross-sectional shape is circular. The cross-sectional size of the shaft, along with the material, are designed to provide sufficient stiffness for predictable control when the finger loop 117 is moved. The shaft slides through an opening in the end of the cylinder body. The interface between the shaft and the opening in the end of the cylinder body is formed to allow for smooth forward and backward movement of the shaft and preferably, at the same time, to provide a waterproof seal.

Another embodiment of the invention includes a control portion 110. In this embodiment, the user grasps the control portion 110 much in the same way that a motorcycle driver grasps the handles of a motorcycle. The user may turn the handles, push them in, pull them out, pivot them about their axes, or, with the aid of a thumb loop, squeeze them. As detailed below, each of these motions creates a corresponding motion at the distal end 120 of the device.

In various embodiments, the control portion 110 is clamped to an object, such as a bed, a table or a cart. In another embodiment, the control portion 110 is clamped to the user's arms or hand. In a further embodiment, the control portion 110 is held by the user, without it being clamped to anything.

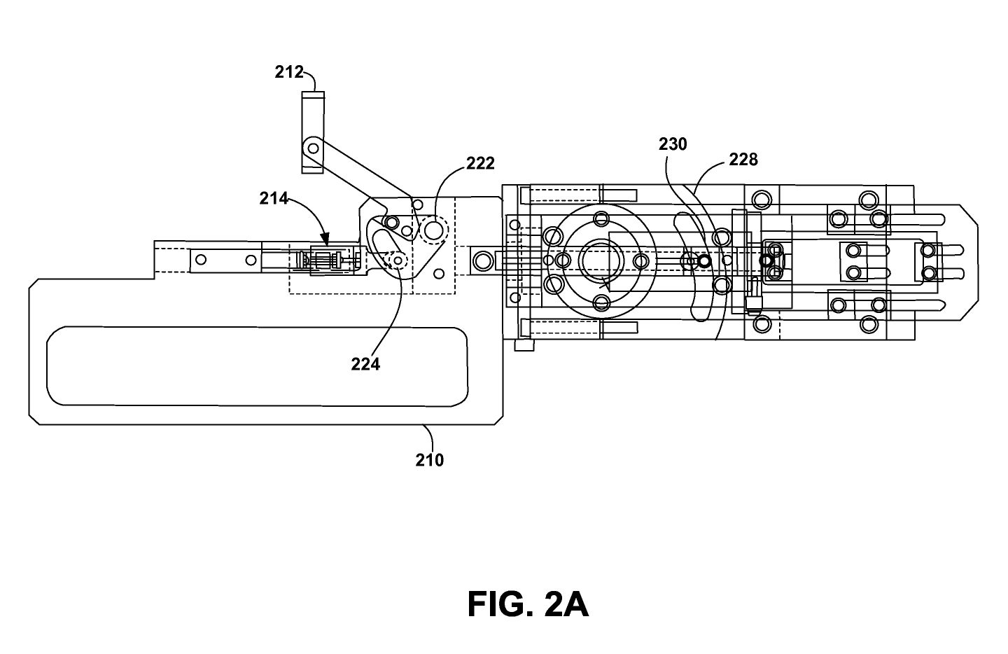

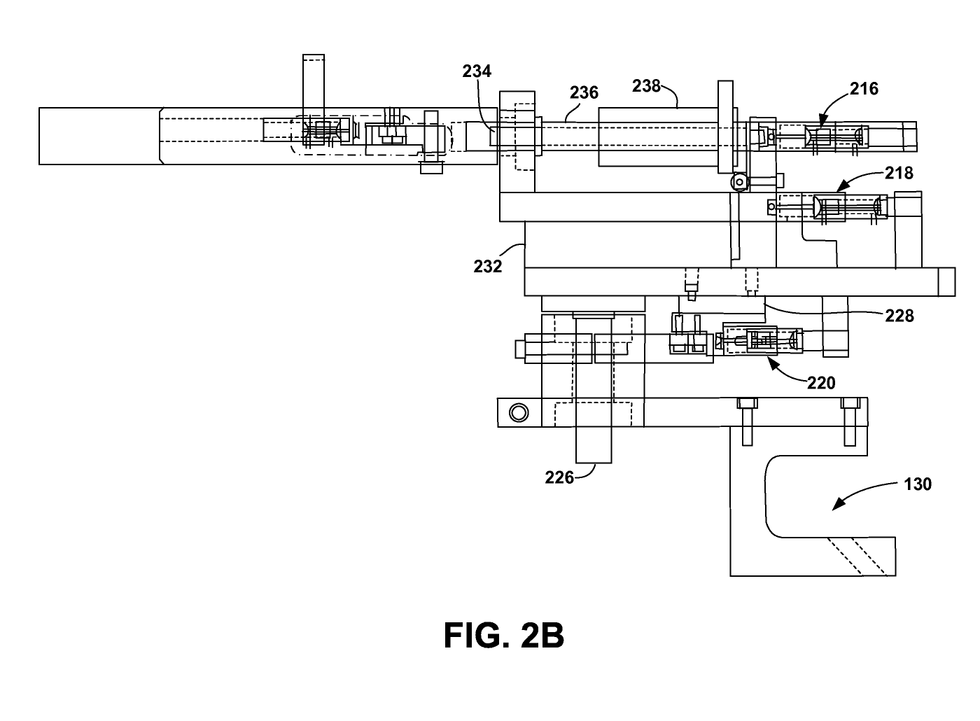

FIG. 2A shows the top view of the control portion 110. A handle 210 is provided for the user's fingers to pass through, while the user's thumb is inserted through a thumb loop 212.

The movements of the control portion 110 are translated into hydraulic motion through the use of control cylinders 214, 216, 218, 220. When the user squeezes the thumb loop 212 towards the handle 210, a bend cam 222 is turned about a vertical axis. As the bend cam 222 turns, a roller 224 is pushed towards the back of the handle. The roller 224 is connected to an outer cylinder 312 of a control cylinder 214 via a shaft 318. The backward movement of the shaft 318 extends a piston 320 backwards, thereby creating the hydraulic pressure needed to actuate a slave cylinder in the distal end 120 of the device. The function of a control cylinder and its connection to a slave cylinder are discussed in greater detail below. In one embodiment of the invention, the squeezing of the thumb loop actuates a grasp function at the distal end 120.

The control portion 110 may be attached to the side of a surgical bed using a clamp 130. However, the control portion is free to rotate about a vertical axis 226, shown in FIG. 2B. The rotation of the control portion 110 about the axis 226 causes a roller 230 to move within a bend cam 228. The roller 230 is connected to an outer cylinder 312 of a control cylinder 220 via a shaft 318. The forward movement of the shaft 318 extends the piston 320 forward, thereby creating the hydraulic pressure needed to actuate a slave cylinder in the distal end 120 of the device. In one embodiment, the turning of the handle results in a rotation of the distal end 120 of the device through a rotate module, described in detail below.

A user may also push the handle 210 forward, in which case, the top portion of the control portion 110 moves forward over a slide 232. The slide 232 is connected to an outer cylinder 312 of a control cylinder 218 via an attachment point 330. The outer cylinder 312 is in turn attached to the piston 320 via a shaft 318. The forward movement of the shaft 318 extends the piston 320 forward, thereby creating the hydraulic pressure needed to actuate a slave cylinder in the distal end 120 of the device. In one embodiment, the forward movement of the handle results in an extension of the distal end 120 of the device through an extension module, described in detail below.

The handle part of the control portion 110 may also rotate along a longitudinal axis coinciding with the shaft 234, as shown in FIG. 2B. In various embodiments, the turning of the handle part causes a screw 236 to rotate within a nut 238. In some embodiments, the screw 236 is stationary and the nut 238 is mobile, whereas in other embodiments of the invention, the screw 236 is mobile and the nut 238 is stationary. The movement of the screw 236 within the nut 238 causes the mobile unit to move linearly with respect to the stationary unit. The mobile unit, whether the screw or the nut, is connected to an outer cylinder 312 of a control cylinder 216 via an attachment point 330. The outer cylinder 312 is in turn attached to the piston 320 via a shaft 318. The forward movement of the shaft 318 extends the piston 320 forward, while the backward movement of the shaft 318 pulls the piston 320 backward. The forward and backward motion of the piston 320 creates the hydraulic pressure needed to actuate a slave cylinder in the distal end 120 of the device. In some embodiments, rotation of the handle part results in the rotation of the distal end 120 of the device through a rotation module, described in detail below.

In certain embodiments of the invention, the movements of the different parts of the control portion 110 creates electrical signals that are sent through wires in the intermediate portion 190 to the slave cylinders in the distal end 120 of the device. The electrical signal is sufficient to actuate a motor in the corresponding slave cylinder, which in turn results in the slave module being actuated. Thus, for example, a forward movement of the handle 210 creates an electrical signal that actuates a motor in an extend module, which results in the extension of that module. Similarly, the rotation of the handle 210, the bending of the handle 210, and the squeezing of the thumb loop 212, result in the rotate module, the bend module, and the grasp module, respectively, being actuated. The slave modules having a motor are described in greater detail below.

Cylinders 214, 216, 218, and 220 are control cylinders. A typical control cylinder 310 is shown in its retracted position in FIG. 3A and in its extended position in FIG. 3B. The control cylinder 310 comprises an outer cylinder 312 and an inner cylinder 314. The inner cylinder 314 has a diameter that allows it to move within the outer cylinder 312. The outer cylinder 312 is connected to a shaft 318, which in turn is connected to the control portion 110 through the attachment point 330. The movements of the control portion 110, described above, causes the outer cylinder 312 to move longitudinally with respect to the stationary inner cylinder 314.

A piston 320, attached to a shaft 318, moves within the inner cylinder 314, within a distance defined by the two inlet points 322, 324 for the hydraulic fluid. The distal end of the shaft 318 is configured to be capable of attachment to the piston 320, while the proximal end of the shaft 318 is configured to be capable of attachment to the outer cylinder at a site close to the attachment point 330. The outer cylinder or the handle assembly may be provided with ratchet teeth. The ratchet teeth are adapted to engage with a locking mechanism to secure the piston 320 at a desired position relative to the cylinder body. Alternatively, a locking mechanism may employ a friction lock to secure the piston 320 at a desired position.

The piston 320 has a solid front face and is movable along the longitudinal axis of the inner cylinder 314. The front face of the piston 320 is identical in shape to the cross section of the cylindrical cavity. The outer surface of the piston 320 forms an airtight seal with the inner surface of the inner cylinder 314. Thus, the portion of the cavity on one side of the piston 320 does not communicate with the portion of the cavity on the other side of the piston 320. At the same time, the piston 320 must be allowed to move smoothly back and forth along the longitudinal axis of the inner cylinder 314.

The proximal end of the inner cylinder 314 is sealed with a seal 316, comprising an opening therethrough, through which the shaft 318 can slide. The distal end of the inner cylinder 314 is sealed with another seal 328, optionally comprising an O-ring 326.

Thus, in the extended position of the control cylinder 310, FIG. 3B, the piston 320 is at rest against the proximal seal 316. The hydraulic fluid is located in the inner cylinder 314 in front of the piston 320. When the control portion 110 is moved in a way described above, i.e., when the handle 210 is moved forward, the outer cylinder 312 moves forward, thereby moving the shaft 318 and the piston 320. Hydraulic fluid exits the inner cylinder 314 through an inlet 324, creating a hydraulic pressure at a point in the distal end 120 of the device. Additional hydraulic fluid, displaced from a slave cylinder, enters to the back of the piston 320 through another inlet 322, thereby keeping the volume of the hydraulic fluid in the system constant. When the control portion 110 is moved completely, the control cylinder 310 is in its retracted position, FIG. 3A. In this position, the piston 320 is at the distal end of the inner cylinder 314, resting against the distal seal 328. The hydraulic fluid is in the back of the piston 320. Those of skill in the art understand that although in the above discussion the piston 320 is described to move from the fully retracted position to the fully extended position, the piston 320 may move from any point along the two extremes to any other point along the two extremes, and thereby cause a corresponding movement in a slave cylinder.

The cannula 190 comprises hydraulic tubings, connecting the control cylinders of the control portion 110 with the slave cylinders at the distal end 120, and housings for the hydraulic tubings.

The distal end 120 comprises modular components. The components can be selected from, for example, an extend module, a rotate module, a bend module, and a grasp module. Other functions can be included as well and activated in the manner described in detail below. Each module is individually describe in greater detail below. The invention is adapted such that the user can pick the combination of modules and the quantity of each individual module that is best suitable for the user's needs and assemble them conveniently.

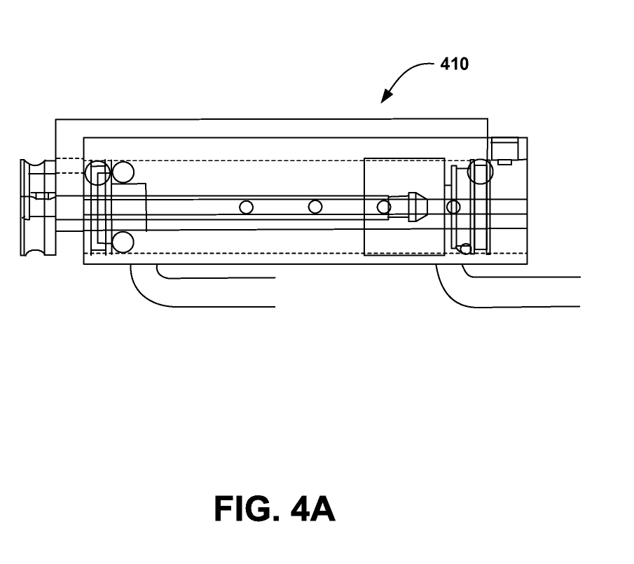

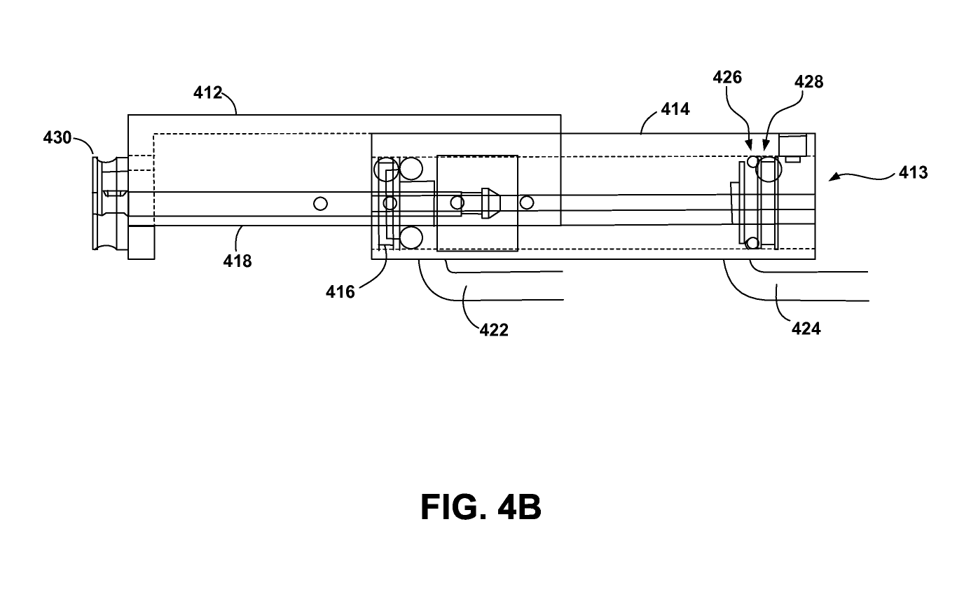

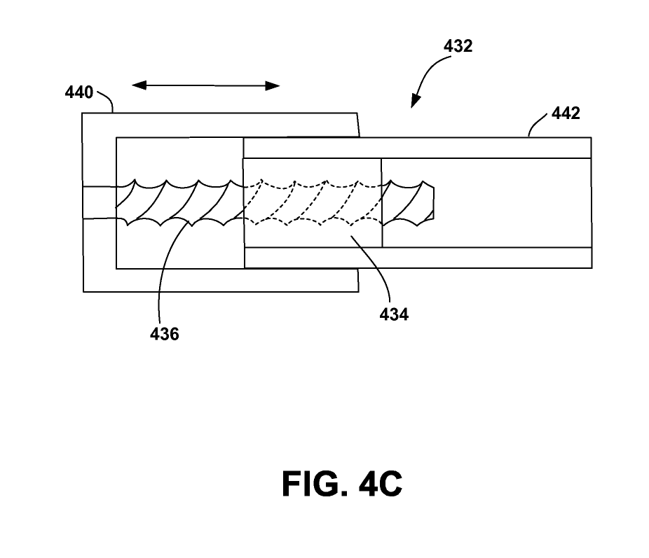

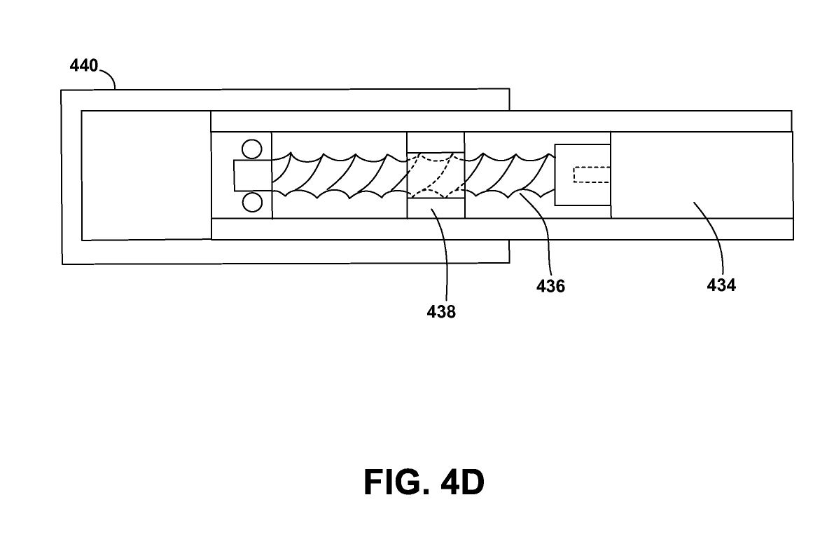

The extend module 410 is depicted in both its retracted position, FIG. 4A, and extended position, FIG. 4B. The extend module 410 is identical in its construction to the control module 310; however, the function of the two are reversed. By applying hydraulic pressure using the control portion 110, hydraulic fluid enters the inner cylinder 414 pushing the piston 420 towards the distal end of the module and the distal seal 416. The shaft 418 moves through the distal seal 416, but it is attached to the outer cylinder 412 at the distal end of the outer cylinder 430. The movement of the piston 420 moves the outer cylinder 412 towards the distal end of the module, thereby extending the cannula. The hydraulic fluid present inside the inner cylinder 414 exits the inner cylinder 414 through the distal outlet 422. The proximal seal 428 prevents the leakage of hydraulic fluid from proximal end of the inner cylinder 414.

Additional modules can be attached to the extend module either at its distal end, through the distal attachment point 430, or at its proximal end, through the proximal attachment point 431.

In another embodiment, the extend module may be extended using electrical power instead of hydraulic power. In this embodiment, by pushing forward on the handle 210 of the control portion 110, the user causes an electrical connection to be formed, whereby electrical signal is sent from the control portion 110 through wires in the intermediate portion 190 to the extend module 432, as depicted in FIGS. 4C and 4D. The electrical signal causes an electrical motor 434 to turn. In one embodiment, as depicted in FIG. 4C, a screw 436 is mounted within the motor 434. The turning of the motor 434 causes the screw to move outward, thereby causing the outer cylinder 440 to move away from the inner cylinder 442. In this embodiment, the motor is stationary, i.e., it is attached to the inner cylinder 442, whereas the screw is mobile, i.e., it moves with respect to the motor and the inner cylinder 442. The screw 436 is attached at its distal end to the outer cylinder 440.

In another embodiment, as depicted in FIG. 4D, the motor 434 causes the screw 436 to turn within a nut 438. The nut 438 is attached to the outer cylinder 440. The turning of the screw 436 causes the nut 438 to move with respect to the screw 436, thereby moving the outer cylinder 440 longitudinally with respect to the inner cylinder 442, causing the module to extend. In this embodiment, the motor 434 and the screw 436 are stationary with respect to the inner cylinder 442, whereas the nut 438 and the outer cylinder 440 are mobile.

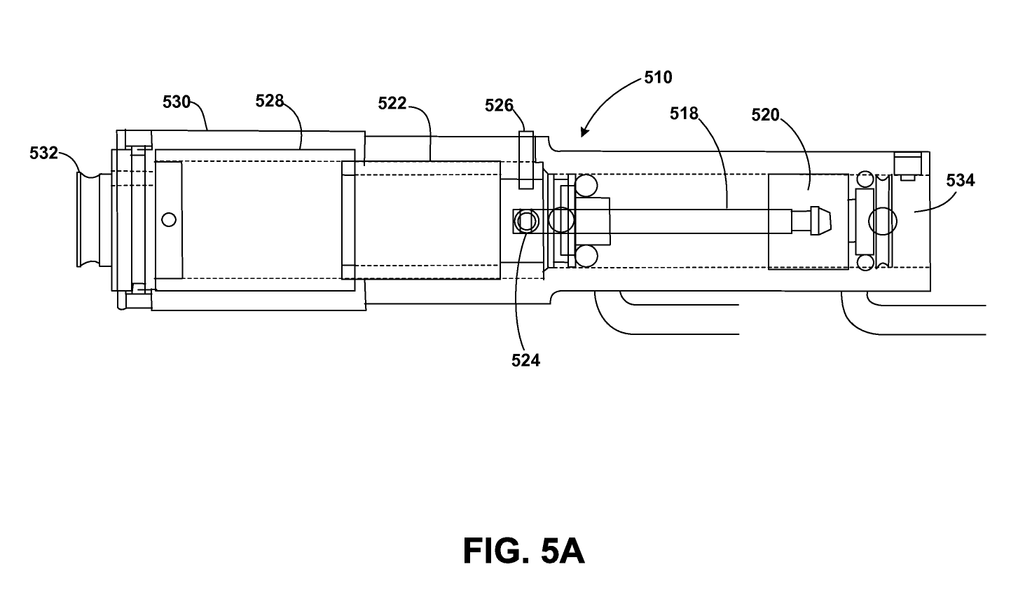

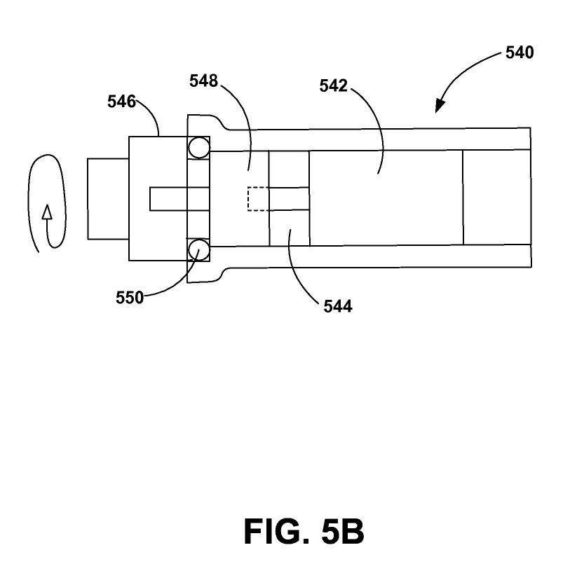

The rotate module 510, as depicted in FIG. 5A, comprises similar hydraulic components as those of the extend module 410. As in the extend module 410, hydraulic pressure, applied by rotating the control portion 110 along a longitudinal axis, causes piston 520 to move toward the distal end of the module, causing the shaft 518 to move in that direction as well. The shaft 518 is attached to a lead screw 522 at an attachment point 524. Extension of the shaft 518 causes the lead screw 522 to move towards the distal end of the module. The lead screw is incapable of rotating, since a stabilizer 526 prevents its rotation. The lead screw 522 instead is extended through a nut assembly 528 which is immovably attached to an outer cylinder 530. The movement of the lead screw 522 through the nut assembly 528 causes the nut assembly 528 to rotate, thereby rotating the outer cylinder 530.

Additional modules can be attached to the rotate module either at its distal end, through the distal attachment point 532, or at its proximal end, through the proximal attachment point 534.

In another embodiment, the rotate module may be rotated using electrical power instead of hydraulic power. In this embodiment, by turning the handle 210 of the control portion 110, the user causes an electrical connection to be formed, whereby an electrical signal is sent from the control portion 110 through wires in the intermediate portion 190 to the rotate module 540, as depicted in FIG. 5B. The electrical signal causes an electrical motor 542 to turn. The electrical motor 542 is attached to a shaft 544 which in turn is attached to the outer cylinder 546. The turning of the shaft rotates the outer cylinder. In some embodiments, a gear reducer assembly 548 may also be present to reduce the rotation speed. In certain embodiments, the connection between the outer cylinder 546 and the cylinder housing the motor assembly 542 may feature a bearing assembly 550.

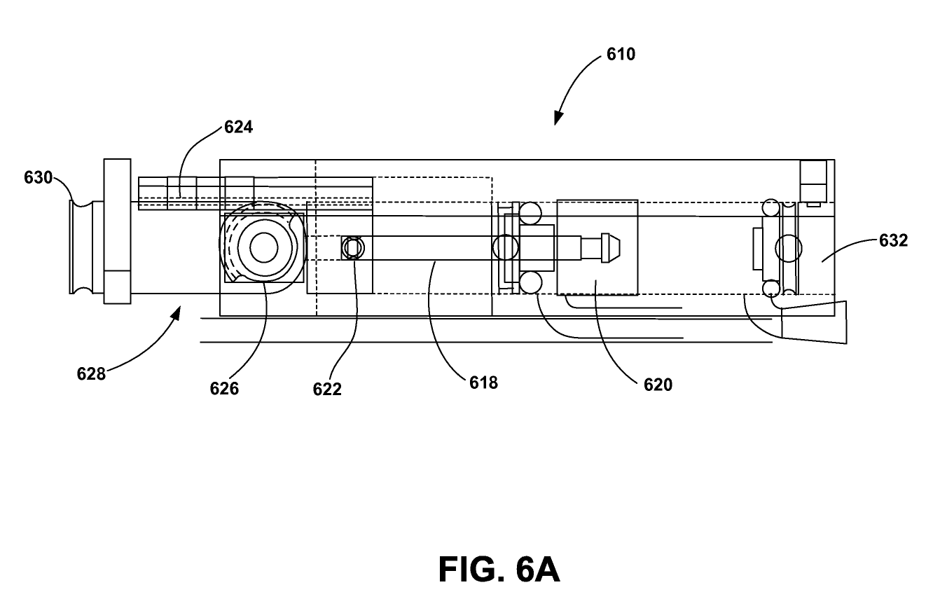

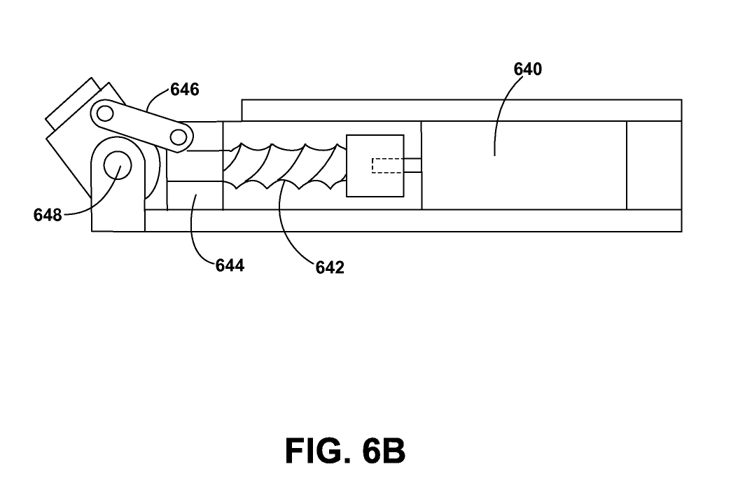

The bend module 610 is depicted in FIG. 6A. This module also features the same hydraulic assembly present in the extend and the rotate modules, above. Applying hydraulic pressure by rotating the control portion 110 along the vertical axis 226 in a clockwise direction causes the piston 620 and the shaft 618 to move towards the distal end of the module. The shaft 618 is attached to a rack 624 either directly or through an attachment assembly 622. The movement of the shaft 618 moves the rack 624. The rack 624 has teeth that correspond to the teeth on a gear 626. The movement of the rack 624 causes the gear 626 to rotate clockwise. The gear 626 is connected to the distal end 628 of the module. The rotation of the gear 626 causes the distal end 628 of the module to bend clockwise. By rotating the control portion 110 in a counter-clockwise direction, the piston 620 is moved towards the proximal end of the module, causing the rack 624 to move backwards as well, which in turn causes the gear 626 to turn counter-clockwise, which in turn causes the distal end 628 of the module to bend counter-clockwise.

In some embodiments, the bending of the distal end 628 of the module is through an angle of at least 110°, i.e., when the piston 620 moves from the proximal end of the hydraulic portion completely to the distal end of the hydraulic portion, the distal end 628 of the module bends at least 110°. In other embodiments, the rotation is an angle of at least 110°, at least 150°, at least 200°, at least 250°, at least 300°, or an angle of at least 350°.

Additional modules can be attached to the bend module either at its distal end, through the distal attachment point 630, or at its proximal end, through the proximal attachment point 632.

In another embodiment, the bend module may be bent using electrical power instead of hydraulic power. In this embodiment, by turning the handle 210 of the control portion 110, the user causes an electrical connection to be formed, whereby electrical signal is sent from the control portion 110 through wires in the intermediate portion 190 to the bend module. The electrical signal causes an electrical motor to turn. The electrical motor is attached to a shaft which in turn is attached to the rack 624. The movement of the shaft 618 moves the rack 624, which in turn causes the gear 626 to rotate, which in turn causes the distal end 628 of the module to bend.

In another embodiment, as depicted in FIG. 6B, the turning of the motor 640 causes a lead screw 642 to rotate within a nut 644. The lead screw 642 is stationary with respect to the motor 640 and the outer body of the module, whereas the nut 644 is mobile. The nut 644 is connected to a link 646 at the proximal end of the link 646. The distal end of the link 646 is connected to the distal end of the module. When the nut 644 is moved backwards, it causes the link 646 to move backwards, thereby causing the distal end of the module to rotate. Reversing the electrical current, by rotating the control portion 110 in the opposite direction, will cause the motor to turn in the opposite direction, thereby causing the nut to move forward and the distal end of the module to bend in a clockwise direction.

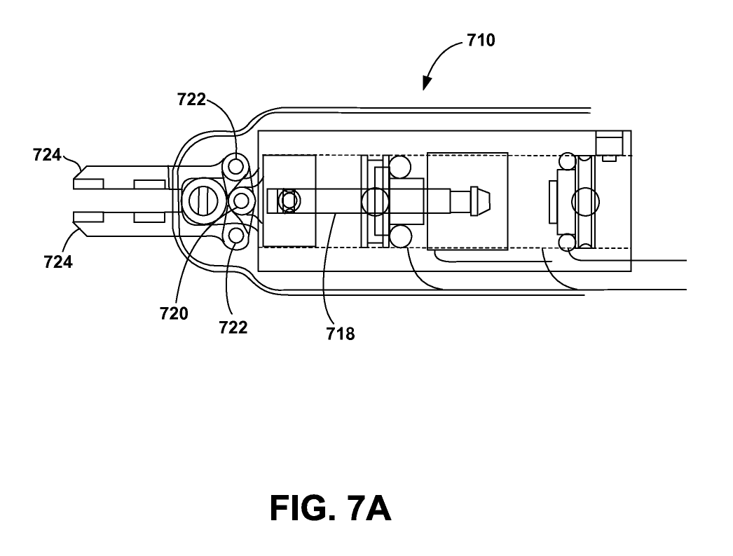

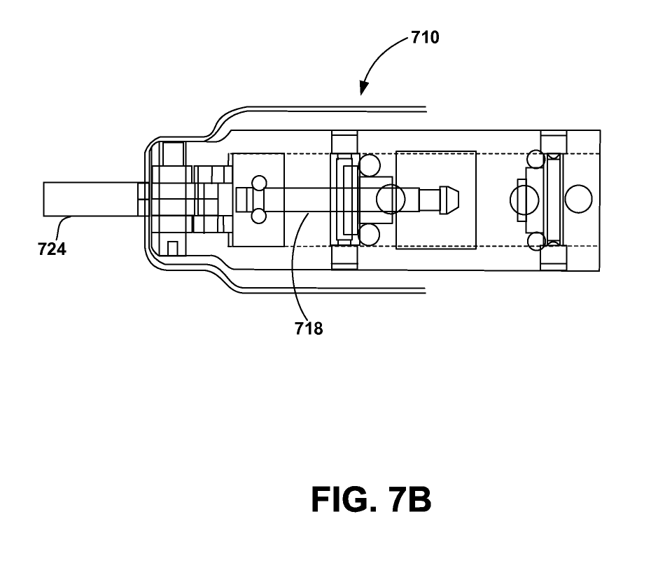

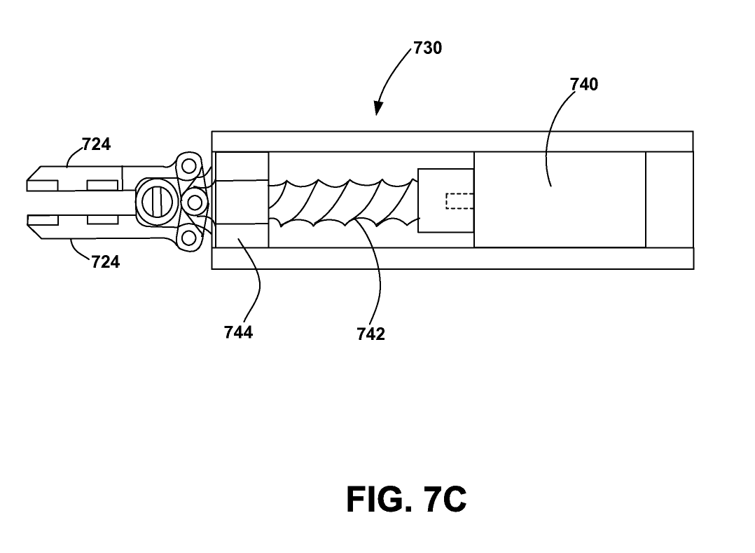

FIG. 7A depicts the top view of the grasp module 710 and FIG. 7B depicts its side view, in accordance to embodiments of the present invention. The grasp module 710 also features a hydraulic portion similar to those of other modules. When the thumb loop 212 is squeezed towards the handle 210, hydraulic pressure is applied and the shaft 718 moves towards the distal end of the module. This movement causes the pin 720 to move towards the distal end of the module as well, thereby causing the two pins 722 to move away from the center. As the two pins 722 move away from the center, the angle defined by pin 722-pin 720-pin 722 tends away from 90° and towards 180°. The movement of the pins 722 causes the two tynes 724 to move towards each other and, eventually, touch. Moving the thumb loop 212 away from the handle 210 will have the opposite effect of causing the tynes 724 to move away from each other and open up.

In another embodiment, the squeezing of the thumb loop 212 causes an electrical current to turn a motor 740, as depicted in FIG. 7C, in the grasp module 730. The motor 740 turns a stationary lead screw 742, which in turn causes a nut 744 to move longitudinally. The movement of the nut 744 causes the tynes to move closer to each other and, eventually, touch. Moving the thumb loop 212 away from the handle 210 will have the opposite effect of causing the tynes 724 to move away from each other and open up.

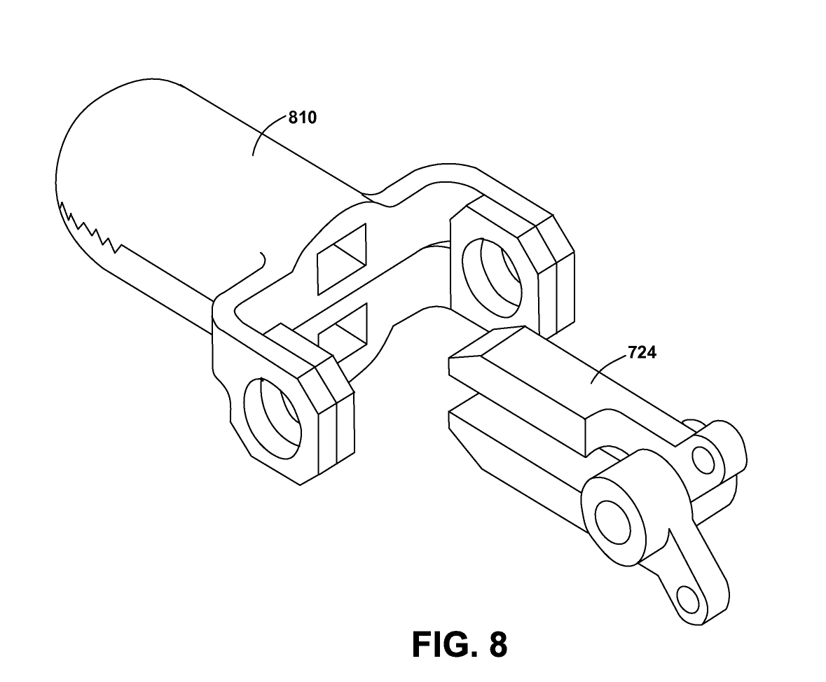

The tynes 724 of the grasp module 710 are configured to accommodate a number of different tools. For example, as depicted in FIG. 8, a grasp tool 810 is shown that can fit over the tynes 724. When the tynes 724 move towards each other, the end portion of the grasp tool 810 also move toward each other and, eventually, touch. If an object or tissue is located between the end portions of the grasp tool 810, the object is then grasped by the tool. There may be a number of tools that can be attached over the tynes 724. In addition to the grasp tool, these include a scissors, a knife for cutting the tissue, drill bits for drilling into bones, heating elements for cauterizing tissue, or any other tool necessary during a surgical procedure.

All the above tools and other tools can fit individually and interchangeably on the grasp module 710. Therefore, during a surgical procedure, the user may attach one tool to the grasp module 710, use it, remove it, and then attach another tool to the same grasp module 710. This process can be repeated any number of times with any number of tools.

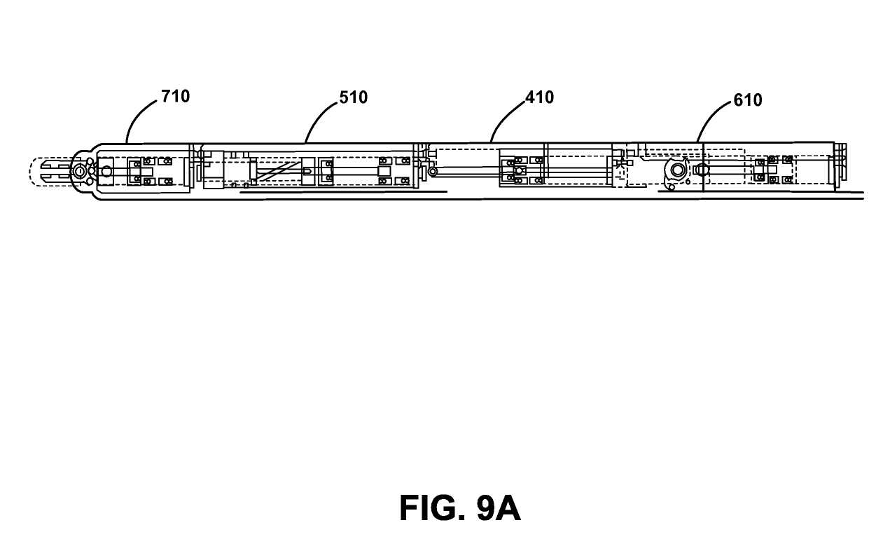

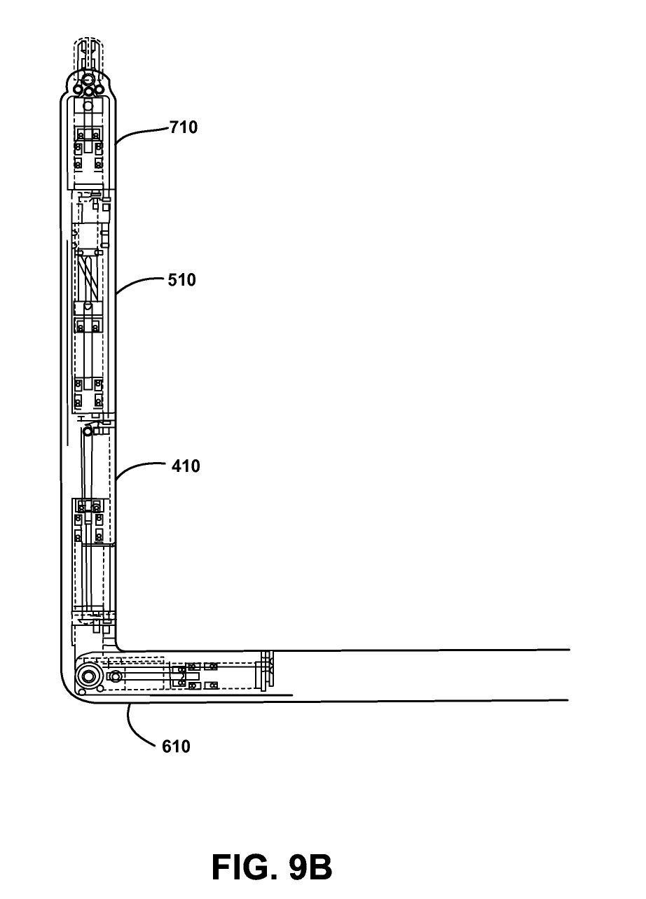

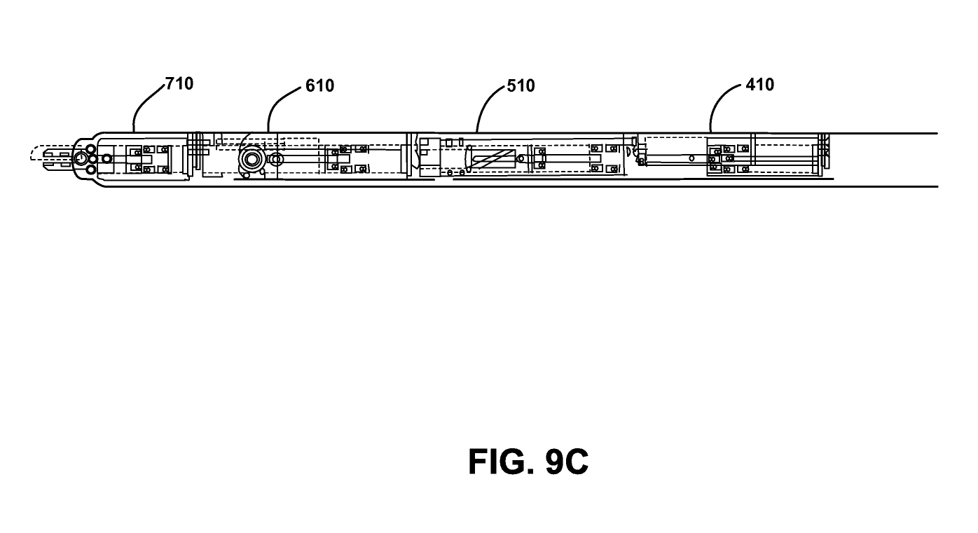

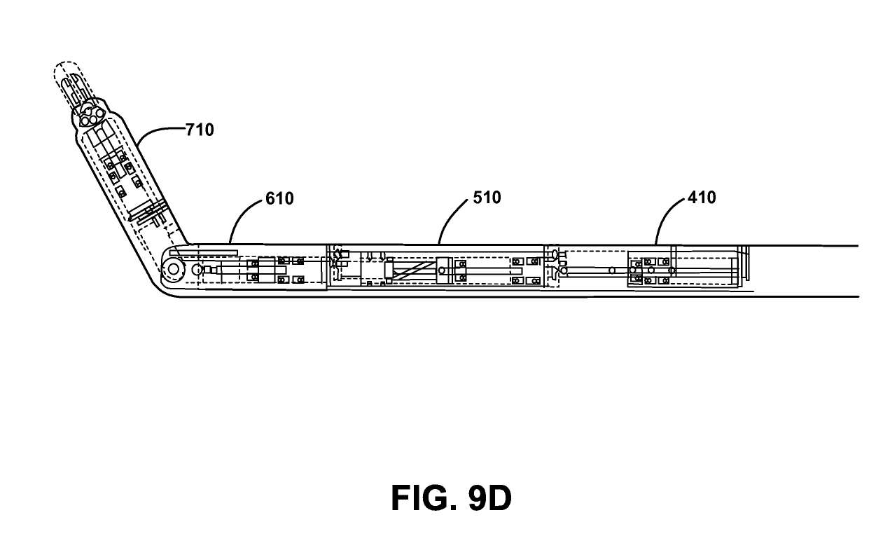

As mentioned above, the modules of the present invention are designed to be placed in order that the user deems most useful. For example, FIG. 9A depicts four of the modules attached in the order of (from proximal end to distal end) bend, extend, rotate, and grasp. FIG. 9A shows the bend module in its retracted position, where the cannula is straight. FIG. 9B shows the bend module in its bent position where the module is bent. Alternatively, the four modules could be arranged in the extend-rotate-bend-grasp configuration, as shown in FIGS. 9C and 9D. Other combinations are also possible. In addition, the user may attach more than a single module of a particular type, for example, two or three or more extend modules or two or three or more bend modules, could be put together, along with other modules to form the distal end 120 of the device. In various embodiments, the grasp module 710 is the most distally located module.

Embodiments of the invention include surgical devices and components coupled with surgical devices. It is appreciated that the surgical devices and other components described in conjunction with the present invention may be electrically, mechanically, hydraulically, directly, indirectly and remotely coupled. It is appreciated that there may be one or more intermediary components for coupling components that may or may not be described.

For example, telemanipulation and like terms such as “robotic” refer to manipulating a master device and translating movement or force applied at the master device into commands that are processed and transmitted to a slave device that receives the commands and attempts to generate the intended movements at the slave device. It is appreciated that when using a telemanipulation device or environment, the master and slave devices can be in different locations.

Embodiments of the present invention are well suited to be used with both telemanipulation systems direct manipulation systems.

In one embodiment, embodiments of the present invention described above may further comprise an end effector coupled to the output end of the plurality of couplings, wherein the end effector moves in response to receiving at least the portion of the input force transmitted by the plurality of couplings. For example, the end effector is grasp module 710 or the combination of grasp module and grasp tool 810. Optionally, the end effector comprises a surgical tool. For example, the end effector is grasp tool 810. It is appreciated that the input force may be generated by a direct manipulation device or may be generated by a telemanipulation device.

In yet another aspect, the present invention may further comprise a manually-driven hydraulic drive system having an input mechanism coupled to the input end of the plurality of couplings, wherein the drive system generates the input force, and an end effector coupled to the output end of the plurality of couplings, wherein the end effector comprises a surgical tool and moves in response to receiving at least the portion of the input force transmitted by the plurality of couplings. It is appreciated that the input force may be generated by a direct manipulation device or may be generated by a telemanipulation device.

The present invention relates to flexible wrist-type elements capable of transmitting axial and/or rotational force around corners and bends. For illustrative purposes, these aspects are discussed herein with respect to a surgical application, however, it should be understood that these aspect may equally apply to many other applications, such as robotics, manufacturing, remote controlled operations, etc., and any application where the transmission of axial and/or rotational force around corners and bends is desired.

Aspects of the present invention include features relating to a flexible wrist-type element for surgical-related activities and methods of manufacture and use thereof, including variations having an angularly movable hub housing and a rotatable and operable end effector driven via additional drive train elements that include one or more flexible couplings, such as universal-type joints. Force transmitted via the set of such elements includes, for example, lineal force and rotational force. It is appreciated that the force transmitted may be generated locally or remotely to the output device and it should be appreciated that embodiments of the present invention are well suited to be used in both direct manipulation and telemanipulation environments.

In one variation, aspects of the present invention include a push-pull-rotate (PPR) element that permits the transmission of axial forces and angular torques around corners or bends. The PPR element may include one or more universal joints (e.g., Hooke's joints) or similarly operating mechanisms arranged in series (in a chain-like configuration) and connected to an input and to an output. The PPR element may be contained within a housing. It is appreciated that the input and/or output may be coupled with a remote telemanipulation device or may be coupled to a direct manipulation device and can be used in both direct manipulation environments and telemanipulation environments.

In some embodiments, a guide element is provided to prevent portions of the PPR element from collapsing under compression and to maintain proper form under extension, among other things. Exemplary motion that may be transmitted to the end effector and/or tools via the PPR element may include rotational motion and push-pull or reciprocating motion that may be used, for example, to cause two or more extensions of the end effector to move relative to one another (e.g., to open and close to allow grasping or cutting, and release). It is appreciated that the exemplary motion may be initiated by a direct manipulation or a telemanipulation input force. It is appreciated that the input force to induce the exemplary motion may be generated in a remote location wherein the input device and output device are coupled with a telemanipulation system.

In one variation, the guide element is responsive to the bend angle and is adjusted appropriately or automatically adjusts its position as a function of operation of the device within a motion limiting mechanism, such as a guide track into which an extension from the guide element slides. The bending of the device to various bend angles may be accomplished via use of one or more pivot points and control mechanisms, such as tendon-like linkages. The PPR element may be attached to a source or sources of axial and torsional input (also interchangeably referred to herein as an “input mechanism”), such as a rotatable and extendable and retractable shaft, housed in a body portion. It is appreciated that the source input may be from a direct manipulation or a telemanipulation input force.

Axial and torsional inputs to each of the PPR elements are then transmitted from the PPR elements to any output, such as to permit rotation and operation of an end effector. The end effector may rotate, for example, relative to the PPR element via a sleeve. It is appreciated that the input may be separated from the output by a telemanipulation system where the force is transmitted from the input to the output via a telemanipulation system.

Some variations of the present invention use one or more essentially friction-free or low friction components in the PPR element and guide system, such as rolling-element bearings, which results in relatively high mechanical efficiencies (e.g., as compared to push-pull cables or cable-pulley systems). Other portions of the system relating to movement, such as guide track pins and pivots in some variations, can optionally be replaced with or further include low-friction rolling-element bearings for even smoother action. Appropriate guide track, guide housing, and hub or rotating tip components can comprise non-conductive material to manage the distribution of electrical energy to end-effectors. Any components may be plated with an appropriate anti-friction and/or electrically insulating coating and/or be used with suitable lubricating substance or features.

Conversely or in addition, some portions of the system may be electrically conductive, such as for use in electrosurgery applications. For example the outer housing of the device may be non-conductive, so as to insulate inner conductive portions. The motion transmitting inner portions may be conductive so as to allow electrosurgical current to be delivered to the end effector and/or any tools used therewith, while the outer housing thereby insulates the device. In addition to certain components being conductive, conducting lubricants may also be used to ensure or enhance electrical communication. In some variations, the electrical energy communicated may be of high frequency to enhance communication of the energy across abutting surfaces and lubricants. It is appreciated that in one embodiment, the electrical communication may be generated from a telemanipulation system.

Aspects of the present invention relate to interchangeable tools for use within a closed area. In general, disclosed herein is a holder which comprises one or more tools attached thereto. The holder and the attached tools are so configured that they can be inserted into a closed area and easily manipulated therein. Examples of the closed area include inside the body of a patient, as in during laparoscopic or arthroscopic surgery, or inside of a device or a mechanical object, as in during maintenance or repair of the interior of said device or mechanical object.

In one embodiment, the tools are configured to be attached to the distal end of a manipulator, which itself is configured to receive the tools. The distal end of the manipulator can itself be inserted into the closed area. The distal end of the manipulator can be controlled by an operator at a proximal end, i.e., the end closest to the operator. It is appreciated that in one embodiment, the proximal end and operator may be remote to the distal end may be coupled with a telemanipulation system that allows the operator to provide input forces remotely to the patient.

Within the closed area, the operator can choose a desired tool from a selection of tools on the holder and attach it to the distal end of the manipulator. After the operator has used the tool in a desired fashion, the operator can then return the just-used tool to the holder, obtain a second tool from the holder, attach it to the distal end of the manipulator, and use the second tool. The operator can repeat this process as many times as the operator desires, thereby interchanging the tool used inside the closed area without having the need to withdraw the manipulator from the closed area. In one embodiment, the operator can change tools within the patient from a remote location.

As described in detail, this system is designed for use, for example, in laparoscopic surgery. The tools are various surgical tools used within the patient's body. The tools in the holder are inserted into the body. During surgery, the surgeon can use and exchange tools without the need to remove the manipulator or the tools themselves from the body. This represents a significant improvement over existing methods and devices. It is appreciated that in one embodiment, the operator can change tools within the patient even in the case that the operator is remote to the patient. In this embodiment, a telemanipulation system may be used to couple the input end with the output end.

A “manipulator” as used herein refers to a device that at its proximal end comprises a set of controls to be used by an operator and at its distal end comprises means for holding and operating a tool, referred to herein as the “tool receiving device.” The controls allow the operator to move the tool receiving device within the generally closed or confined area, and operate the tool as intended. The tool receiving device is adapted to receive tools interchangeably and can cause a variety of different tools to operate in their intended purpose. Examples of a manipulator include any of a variety of laparoscopic or arthroscopic surgical tools available on the market for use by surgeons, or the device described in U.S. Pat. No. 6,607,475. The tool receiving device of a manipulator is adapted to enter a generally closed or confined area through a small opening, such as a small hole in a mechanical device or a small incision in a human body. It is appreciated that the proximal end may be remote to the distal end and can be used in a telemanipulation environment.

As used herein, “proximal” refers to the part of the device that remains outside of the closed area, closest to the operator. “Distal” refers to the end inserted into the closed area, farthest away from the operator. The proximal and distal ends are preferably in communication with each other, such as fluid communication, electrical communication, communication by cables, telemanipulation and the like. Such communication can occur, for example, through a catheter or cannula, which houses the lines used for such communication. The catheter or cannula is preferably a tube or other substantially cylindrical hollow object. In some embodiments, the catheter or cannula does not house any lines for communication between the proximal and distal ends. In these embodiments, the catheter or cannula is used for placing an object, located substantially at the distal end of the catheter or cannula, inside the closed area for further manipulation. It is appreciated that the distal and proximal ends may be in communication with the use of a telemanipulation system.

During the operation of the devices described herein, the catheter or cannula (hereinafter referred to simply as “cannula”) is inserted into a generally closed or confined area where the tools are to be used such that its proximal end remains outside the closed area while the distal end remains inside the closed area. In the context of surgical procedures, the cannula is inserted into the patient's body such that its proximal end remains outside the body while the distal end remains inside the body. In one embodiment, the proximal end is remote to the patient. This allows the operator, e.g. a surgeon, to access the interior of the closed area, e.g., a patient's body, using the cannula, thereby eliminating the need for “open” surgical procedures both locally and remotely. Only a small incision is needed to insert the cannula, and the various surgical instruments are inserted, and the procedures performed, through the cannula. The proximal end may be remote to the patient and force applied at the proximal end may be translated using a telemanipulation system that recreates the input force at the distal end.

The instruments or tools described herein are capable of being attached to the distal end of the manipulator in a number of different ways. For instance, in some embodiments the tools are attached magnetically, while in other embodiments the tools may clip on to the distal end of the manipulator. In one embodiment, a telemanipulation system may be used to couple the distal and proximal ends. Additional details on the attachment of the tools is provided below.

The manipulator, which is used to position and maneuver the tools within the confined space, can be a hydraulic, pneumatic, robotic, direct manipulation, telemanipulation, standard surgical, minimal invasive surgery (MIS), electrical, or mechanical device, or a device comprising a combination of any of these systems. Any system that can be used to position and manipulate the tools is contemplated.

### Embodiments of Protective Sheath

A discussion regarding embodiments of protective sheaths is provided below. First, the discussion will describe the structure or components of various embodiments of the protective sheaths. Then the discussion will describe the operational description of the protective sheaths in conjunction with the medical instrument.

In general, a protective sheath is configured to control contamination of a medical instrument when the medical instrument and the corresponding protective sheath are placed within a patient, which will be described in detail below. The protective sheath controls contamination by protecting the medical instrument from excessive fouling and contamination. As a result, cleaning efforts are reduced. Moreover, the protective sheath comprises a high dielectric material for insulating the patient from electrical current when the medical instrument and the protective sheath are placed within the patient.

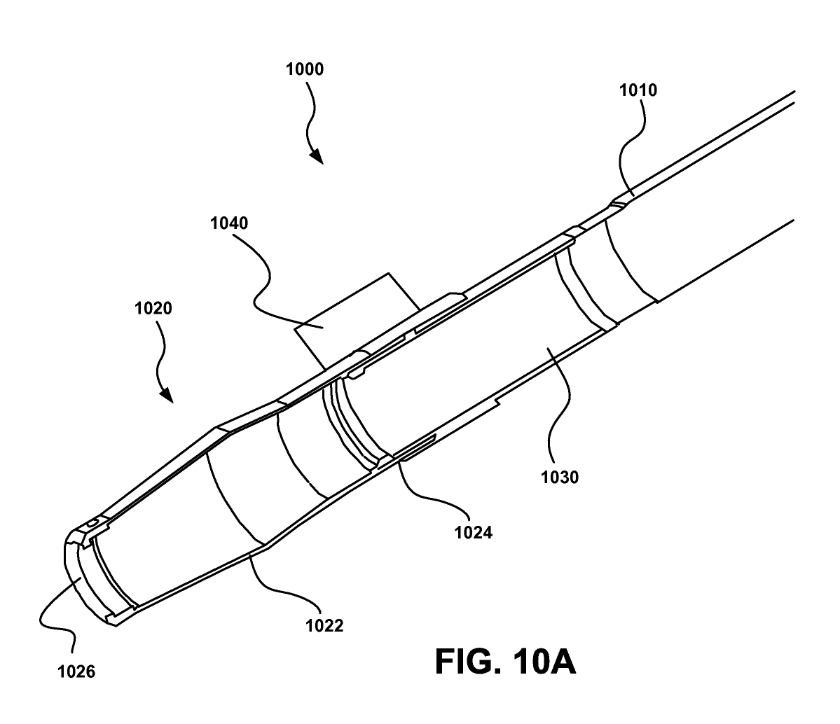

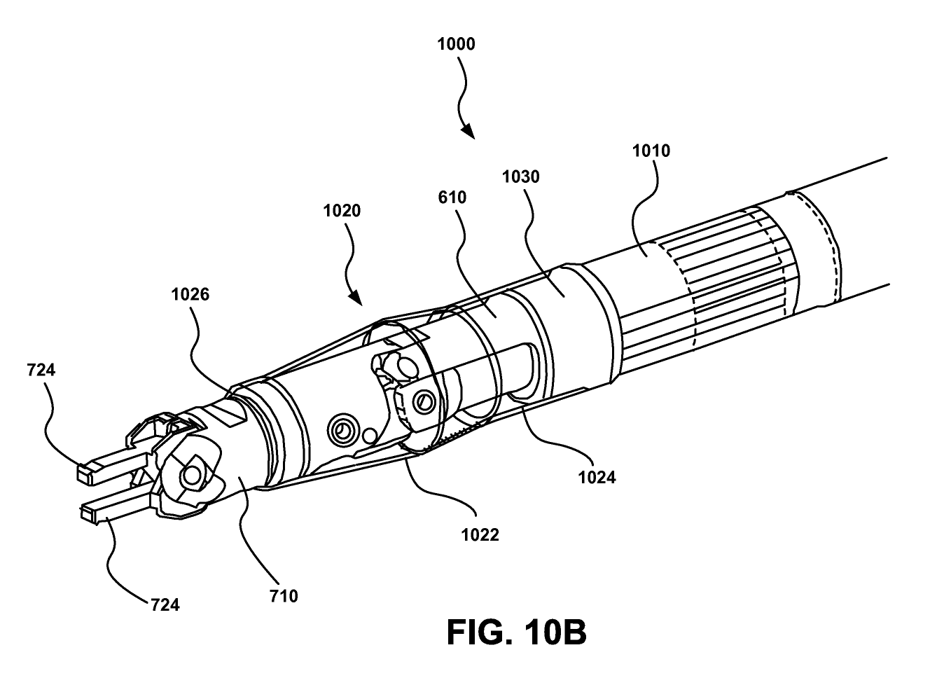

FIGS. 10A-C depict protective sheath 1000, in accordance to embodiments of the present invention. Protective sheath 1000 includes first portion 1010, second portion 1020 and sleeve 1030.

First portion 1010 is configured to receive at least a portion of a medical instrument. In various embodiments, the medical instrument can be, but is not limited to, any combination of the modules, as depicted in FIGS. 1 and 3A-9D and/or any laparoscopic instrument. In one embodiment, first portion 1010 is substantially cylindrical. In various embodiments, first portion 1010 can be any shape that corresponds to the shape of the medical instrument.

Second portion 1020 comprises boot portion 1022 and sub-portion 1024. Second portion 1020 is configured to accommodate movement of a distal end of the medical instrument within second portion 1020. In various embodiments, second portion 1020 is further configured to accommodate rotational, axial and bending movements of the distal end of the medical instrument within second portion 1020. In particular, boot portion 1022 accommodates bending of the medical instrument and sub-portion 1024 accommodates axial and rotational movements of the medical devices, which will be described in detail below.

Second portion 1020 also includes distal aperture 1026. Distal aperture 1026 is configured to allow at least a portion of an end effector of the medical instrument to protrude therefrom.

Sleeve 1030 is configured to facilitate the joining of first portion 1010 and second portion 1020. In one embodiment, sleeve 1030 is rigid.

Sub-portion 1024 of second portion 1020 is joined to sleeve 1030 (via sleeve 1030). In one embodiment, an outer surface of sleeve 1030 seats with an inner surface of sub-portion 1024. In various embodiments, sub-portion 1024 is joined to sleeve 1030 by, but not limited to, overmolding, adhesive, welding and the like. As a result, movement (e.g., translational and/or rotational movement) is not permitted between second portion 1020 and sleeve 1030. Moreover, the joining between sub-portion 1024 and sleeve 1030 is waterproof.

First portion 1010 is joined to sleeve 1030. The joint between first portion 1010 and sleeve 1030 is a waterproof articulating joint that allows for axial and rotational movement. Accordingly, second portion 1020 is axially and rotationally coupled via sub-portion 1024 to first portion 1010. In one embodiment, first portion 1010 is joined to sleeve 1030 via a line fit. In another embodiment, first portion 1010 is reformed over sleeve 1030 to achieve a zero tolerance line fit.

In one embodiment, protective sheath 1000 includes tape clip 1040. Tape clip 1040 is configured to maintain the position, as shown in FIG. 10A, of first portion 1010 with respect to second portion 1020 during installation.

Referring to FIG. 10B, protective sheath 1000 surrounds the medical instrument. Second portion 1020 is shown as being transparent for purposes of clarity. Second portion 1020 surrounds bend module 610. In one embodiment, distal aperture 1026 allows at least a portion of grasp module 710 to protrude. In another embodiment, distal aperture 1026 allows at least a portion of an end effector to protrude.

During use of the medical instrument with corresponding protective sheath 1000, boot portion 1022 accommodates the bending of bend module 610. For example, the bending of bend module 610 occurs within boot portion 1022. In particular, boot portion 1022 flexes and bends in response to the bending of module 610.

Sub-portion 1024 (in conjunction with sleeve 1030) accommodates axial and rotational movements of the medical instrument. Sleeve 1030 axially moves within first portion 1010 in response to the extending/retracting of extend module 410 (not shown in FIG. 10B).

FIGS. 10A-B depict protective sheath 1000 in a retracted position. FIG. 10C depicts protective sheath 1000 in an extended position. For example, as extend module 410 is extended, second portion 1020 slides away from first portion 1010.

In regards to accommodating rotational movement, as rotational module 510 (not shown in FIG. 10B) rotates, sleeve 1030 rotationally moves within first portion 1010. In various embodiments, sleeve 1030 rotates within first portion 1010 at any axial position of sleeve 1030 within first portion 1010. For example, sleeve 1030 is able to rotate within first portion 1010 at any position between (and including) the extended and retraced positions of extend module 410.

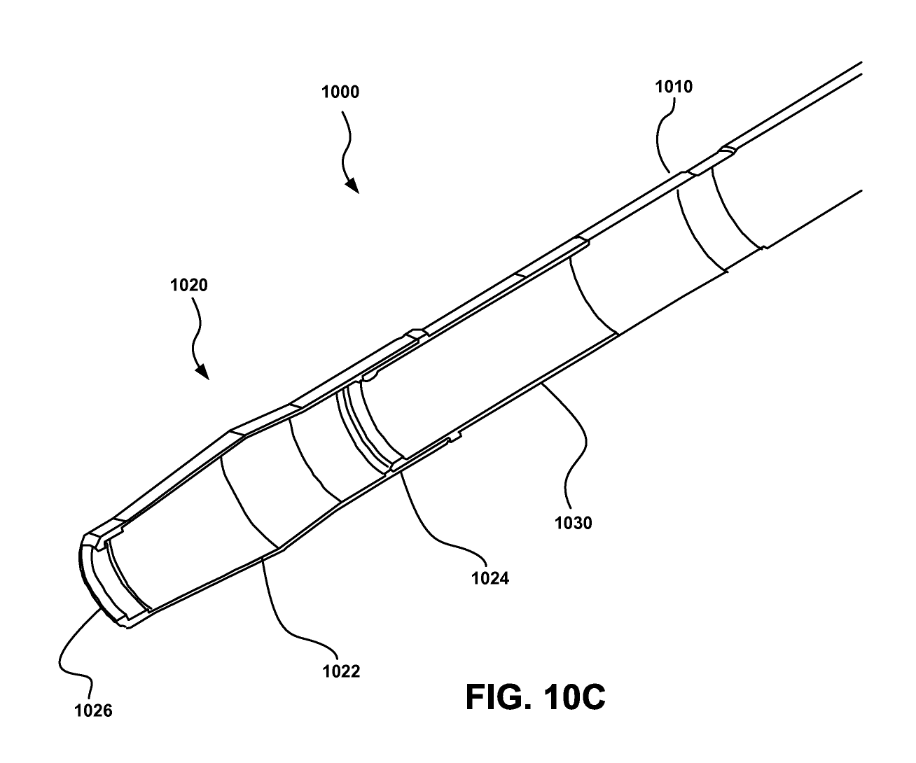

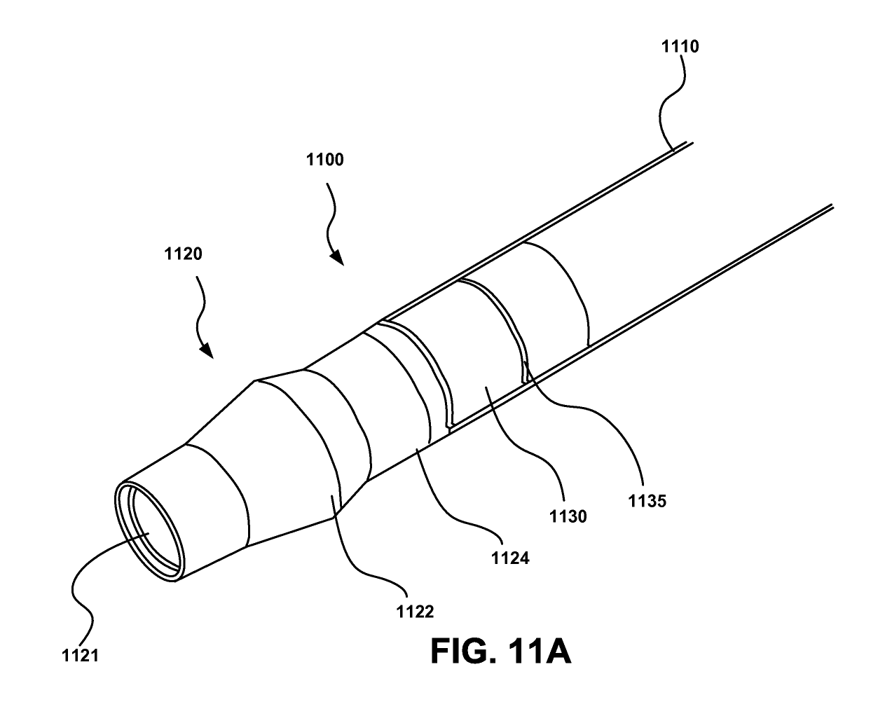

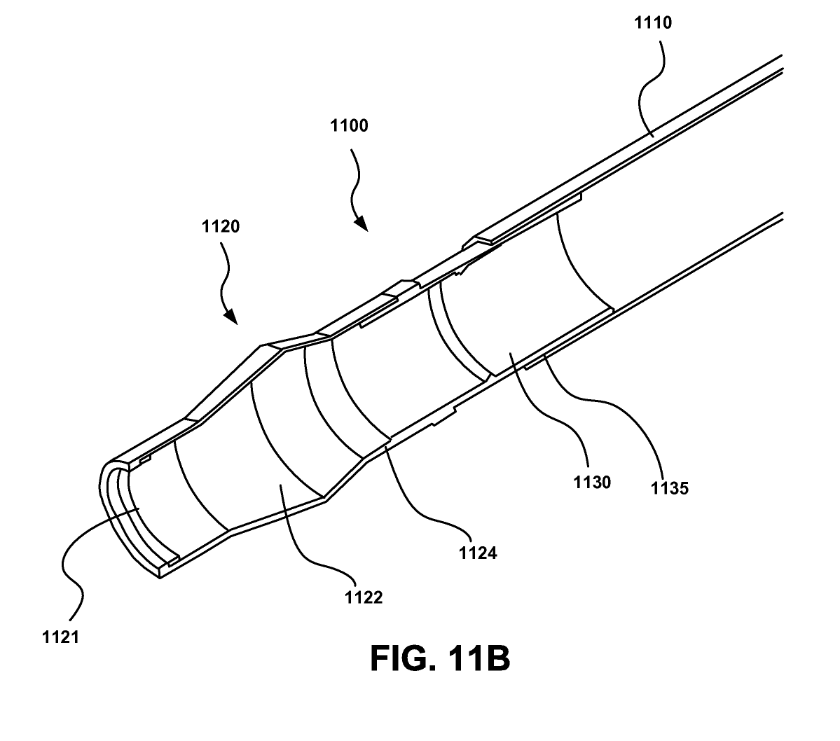

FIGS. 11A-B depicts protective sheath 1100, in accordance to embodiments of the present invention. Protective sheath 1100 includes first portion 1110 (shown as transparent in FIG. 11A), second portion 1120 and sleeve 1130. Protective sheath 1100 is similar to protective sheath 1000, as described above.

First portion 1110 is configured to receive at least a portion of a medical instrument. In one embodiment, first portion 1110 is the same as first portion 1010, as described above.

Second portion 1120 is configured to accommodate movement of a distal end of the medical instrument within second portion 1120. In one embodiment, second portion 1120 is the same as second portion 1020, as described above.

In another embodiment, second portion 1120 includes stiffening ring 1121. Stiffening ring 1121 is configured to stiffen boot 1122.

Sleeve 1130 is configured to facilitate the joining of first portion 1110 and second portion 1120, as described above. Sleeve 1130 includes compliant seal 1135. Compliant seal 1135 is configured to provide a waterproof articulating joint between sleeve 1130 and first portion 1110, which is described in detail below.

Sub-portion 1124 of second portion 1120 is joined to sleeve 1130. In one embodiment, sub-portion 1124 is joined to sleeve 1130 in the same way sub-portion 1024 is joined to sleeve 1030, as described above. Accordingly, movement (e.g., translational and/or rotational movement) is not permitted between second portion 1120 and sleeve 1130. Moreover, the joining between sub-portion 1124 and sleeve 1130 is waterproof.

First portion 1110 is joined to sleeve 1130. The joint between first portion 1110 and sleeve 1130 is a waterproof articulating joint that allows for axial and rotational movement. Accordingly, second portion 1120 is axially and rotationally coupled via sub-portion 1124 to first portion 1110.

During use of the medical instrument, boot portion 1122 accommodates the bending of bend module 610. For example, the bending of bend module 610 occurs within boot portion 1122. In particular, boot portion 1122 flexes and bends in response to the bending of module 610.

Sub-portion 1124 (in conjunction with sleeve 1130) accommodates axial and rotational movements of the medical instrument, as described above.

In regards to accommodating axial movement, as extend module 410 (not depicted in FIGS. 11A-B for purposes of clarity) extends/retracts, sleeve 1130 axially moves within first portion 1110. Specifically, FIG. 11A depicts protective sheath 1100 in a retracted position. FIG. 11B depicts protective sheath 1100 in an extended position. For example, as extend module 410 is extended, second portion 1120 slides away from first portion 1110.

In regards to accommodating rotational movement, as rotational module 510 (not shown in FIGS. 11A-B for purposes of clarity) rotates, sleeve 1130 rotationally moves within first portion 1110. In various embodiments, sleeve 1130 rotates within first portion 1010 at any axial position of sleeve 1130 within first portion 1110. For example, sleeve 1130 is able to rotate within first portion 1110 at any position between (and including) the extended and retraced positions of extend module 410.

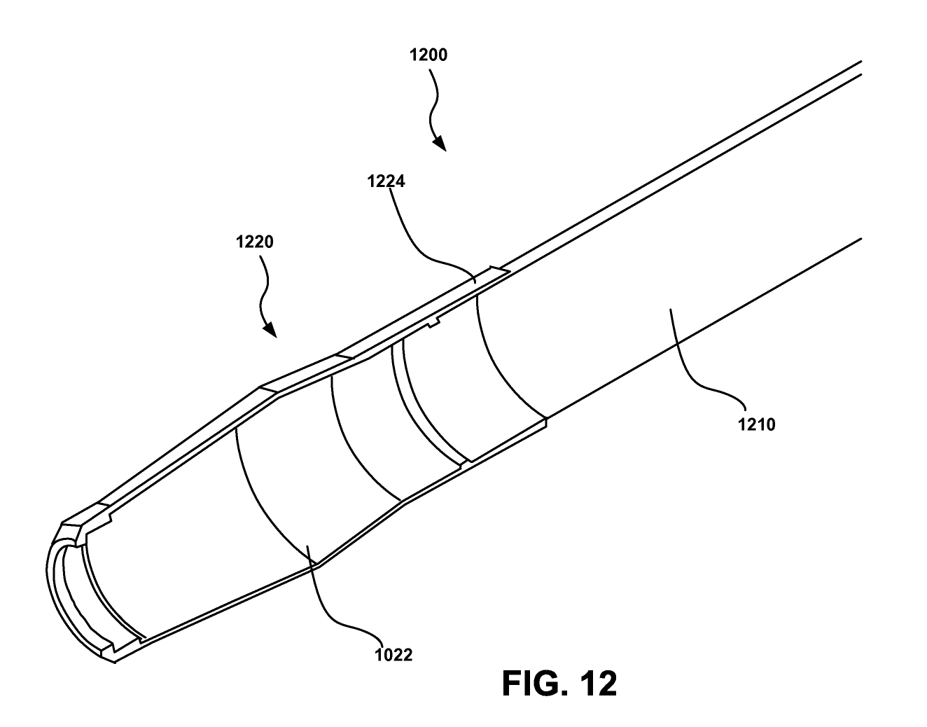

FIG. 12 depicts protective sheath 1200, in accordance to an embodiment of the present invention. Protective sheath 1200 includes first portion 1210 (shown as transparent in FIG. 12) and second portion 1220. Protective sheath 1200 is similar to protective sheaths 1000 and 1100, as described above. However, protective sheath 1200 does not include a sleeve (e.g., sleeve 1030 or 1130).

First portion 1210 is configured to receive at least a portion of a medical instrument. In one embodiment, first portion 1210 is the same as first portion 1010, as described above.

Second portion 1220 is configured to accommodate movement of a distal end of the medical instrument within second portion 1220. In one embodiment, second portion 1220 is the same as second portion 1020, as described above.

Sub-portion 1224 of second portion 1220 is joined to first portion 1210. In one embodiment, an outer surface of first portion 1210 seats with an inner surface of sub-portion 1224. In various embodiments, sub-portion 1224 is joined to first portion 1210 by, but not limited to, overmolding, adhesive, welding and the like. As a result, movement (e.g., translational and/or rotational movement) is not permitted between second portion 1220 and first portion 1210. Moreover, the joining between second portion 1220 and first portion 1210 is waterproof.

During use of the medical instrument, boot portion 1222 accommodates the bending of bend module 610 (not shown in FIG. 12). For example, the bending of bend module 610 occurs within boot portion 1222. In particular, boot portion 1222 flexes and bends in response to bending of bend module 610.

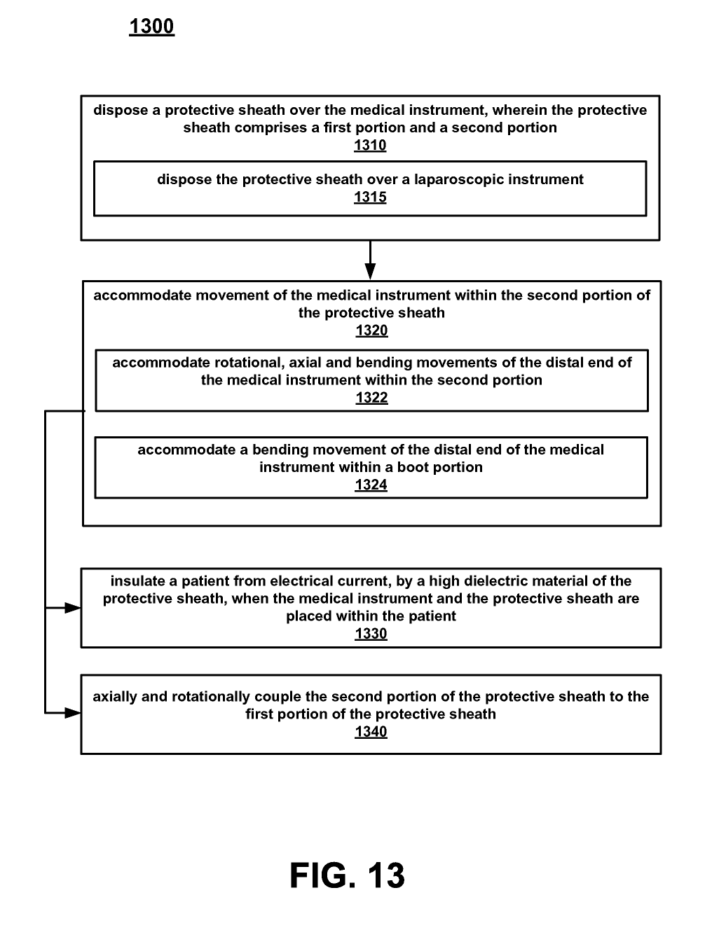

FIG. 13 depicts a method 1300 for protecting a medical instrument, in accordance to an embodiment of the present invention. At 1310 of method 1300, a protective sheath is disposed over the medical instrument, wherein the protective sheath comprises a first portion and a second portion. In one embodiment, at 1315 of method 1300, a protective sheath 1000 is disposed over laparoscopic instrument.

At 1320 of method 1300, movement of the medical instrument is accommodated within the second portion of the protective sheath. In one embodiment, at 1322 of method 1300, rotational, axial and bending movements of the distal end of the medical instrument are accommodated within the second portion. In another embodiment, at 1324 of method 1300, a bending movement of the distal end of the medical instrument is accommodated within a boot portion.

At 1330 of method 1300, a patient is insulated from electrical current, by a high dielectric material of the protective sheath, when the medical instrument and the protective sheath are placed within the patient.

At 1340 of method 1300, the second portion of the protective sheath is axially and rotationally coupled to the first portion of the protective sheath.

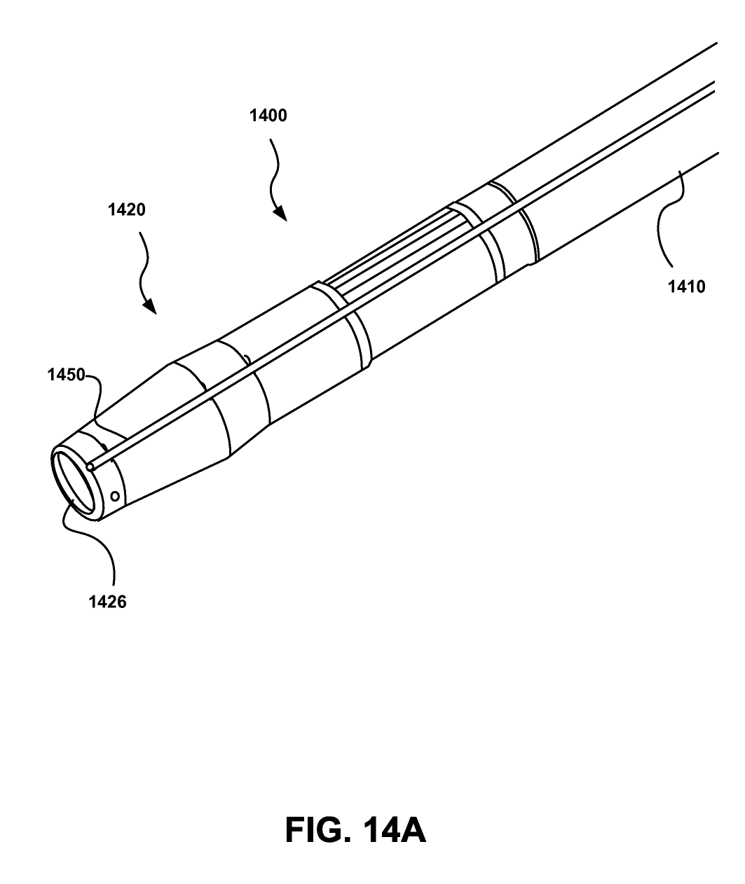

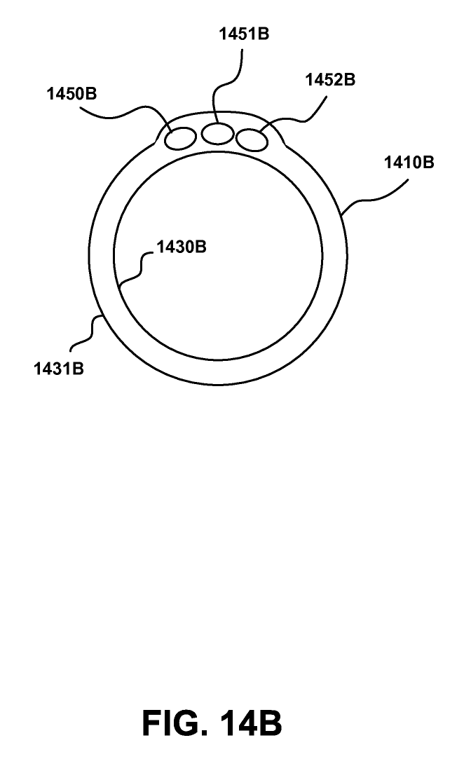

FIG. 14A depicts protective sheath 1400, in accordance to an embodiment of the present invention. In one embodiment, protective sheath 1400 is the same as protective sheath 1000. In particular, first portion 1410 and second portion 1420 are the same as first portion 1010 and second portion 1020, respectively. However, protective sheath 1400 also includes tube 1450 that runs along the length protective sheath 1400. In various embodiments, protective sheaths 1100 and 1200, as described above, also include tube 1450.

Tube 1450 is configured to assist the medical instrument (not shown). In various embodiments, tube 1450 is configured to (but not limited to) provide irrigation, a light source, an electrical source for cauterization, and suction.

In one embodiment, an irrigation fluid (any physiologically compatible fluid) is provided within tube 1450. For example, irrigation fluid is pumped through tube 1450 to irrigate the area in proximity to the distal end of the medical instrument.

In another embodiment, a light source is provided within tube 1450. For example, a light tube, such as, but not limited to a fiber optic cable, can disposed within tube 1450. As a result, light is provided in proximity of the distal end of the medical instrument.

In a further embodiment, an electrical source is provided within tube 1450. For example, an electrical current is provided through tube 1450 for cauterizing the area in proximity of the distal end of the medical instrument.

In another embodiment, suction is provided within tube 1450. For example, suction flows within tube 1450. As a result, fluids are removed from the patient in proximity to the distal end of the medical instrument.

FIG. 14A depicts tube 1450. However, protective sheath 1400 can include any number of tubes in any configuration along the length of protective sheath 1400. In one embodiment, protective sheath 1400 may include a plurality of tubes adjacent one another, in the same orientation as tube 1450. In another embodiment, protective sheath 1400 includes a plurality of tubes evenly or unevenly spaced around the periphery of protective sheath 1400.

FIG. 14B depicts a cross-sectional view of protective sheath 1400, in accordance to an embodiment of the present invention. In this embodiment, protective sheath 1400 includes tubes 1450B-1452B that are disposed between an inner surface 1430B and outer surface 1431B of first portion 1410B. Tubes 1450B-1452B have elliptical cross-sections.

In embodiments with a plurality of tubes, each tube can provide a different function. For example, tube 1450B can provide irrigation, tube 1451B can provide suction and tube 1452B can provide a light source.

Referring again to FIG. 14A, tube 1450 can accommodate the movement of second portion 1420 with respect to first portion 1410. In particular, tube 1450 is resiliently flexible to accommodate bending, axial and rotational movement of second portion 1420 with respect to first portion 1410.

FIG. 14A depicts a distal end of tube 1450 flush with distal aperture 1426. However, in one embodiment, the distal end of tube 1450 may extend beyond distal aperture 1426. For example, distal end of tube 1450 extends to and is connected to an end effector (not shown). In another embodiment, distal end of tube 1450 is recessed from distal aperture 1426.

In one embodiment, tube 1450 is physically connected to first portion 1410. In another embodiment, tube 1450 is not required to be physically connected to second portion 1451.

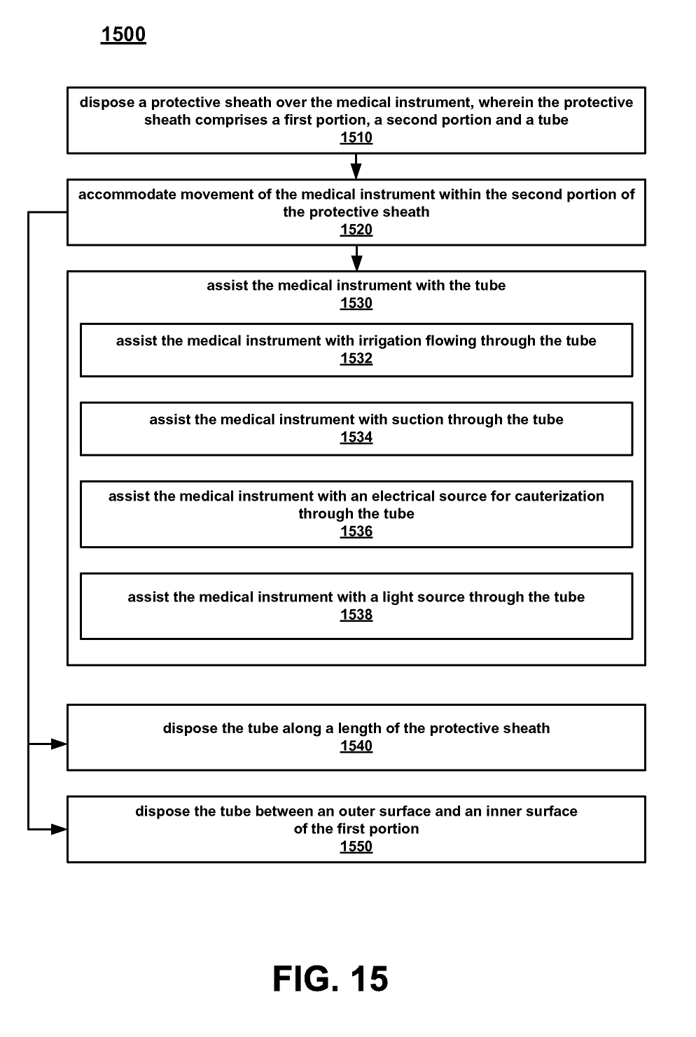

FIG. 15 depicts a method 1500 assisting a medical device, in accordance to an embodiment of the present invention. At 1510 of method 1500, a protective sheath is disposed over the medical instrument, wherein the protective sheath comprises a first portion, a second portion and a tube. At 1520 of method 1500, movement of the medical instrument is accommodated within the second portion of the protective sheath.

At 1530 of method 1500, the medical instrument is assisted by the tube. In one embodiment, at 1532, the medical instrument is assisted with irrigation flowing through the tube. In another embodiment, at 1534, the medical instrument is assisted with suction through the tube. In a further embodiment, at 1536, the medical instrument is assisted with an electrical source for cauterization through the tube. In another embodiment, the medical instrument is assisted with a light source through the tube.

At 1540 of method 1500, the tube is disposed along a length of the protective sheath. At 1550 of method 1500, the tube is disposed between an outer surface and an inner surface of the first portion.

Various embodiments of the present invention are thus described. While the present invention has been described in particular embodiments, it should be appreciated that the present invention should not be construed as limited by such embodiments, but rather construed according to the following claims.

1. A protective sheath comprising:
a first portion configured to receive a medical instrument;
a second portion configured to accommodate movement of a distal end of said medical instrument within said second portion, wherein said second portion is movably coupled to said first portion, and wherein said protective sheath is configured to control contamination of said medical instrument when said medical instrument and said protective sheath are placed within a patient; and
a tube configured to assist said medical instrument when said medical instrument and said protective sheath are placed within said patient, wherein said tube is disposed along a length of said protective sheath.

2. The protective sheath of claim 1, wherein said tube comprises cylindrical cross-section.

3. The protective sheath of claim 1, wherein said tube comprises an elliptical cross-section.

4. The protective sheath of claim 1, wherein said tube is disposed on an outer surface of said protective sheath.

5. The protective sheath of claim 1, wherein said tube is disposed between an outer surface and an inner surface of said first portion.

6. The protective sheath of claim 1, wherein said tube comprises:
a plurality of tubes.

7. The protective sheath of claim 1, wherein said tube is further configured to provide irrigation.

8. The protective sheath of claim 1, wherein said tube is further configured to provide a light source.

9. The protective sheath of claim 1, wherein said tube is further configured to provide an electrical source for cauterization.

10. The protective sheath of claim 1, wherein said tube is further configured to provide suction.

11. The protective sheath of claim 1, wherein said tube is connected to an end effector.

12. A method for assisting a medical instrument, said method comprising:
disposing a protective sheath over said medical instrument, wherein said protective sheath comprises a first portion, a second portion and a tube;
accommodating movement of said medical instrument within said second portion of said protective sheath; and
assisting said medical instrument with said tube.

13. The method of claim 12, wherein said assisting said medical instrument comprises:
assisting a laparoscopic instrument.

14. The method of claim 12, wherein said assisting said medical instrument with said tube comprises:
assisting said medical instrument with irrigation flowing through said tube.

15. The method of claim 12, wherein said assisting said medical instrument with said tube comprises:
assisting said medical instrument with suction through said tube.

16. The method of claim 12, wherein said assisting said medical instrument with said tube comprises:
assisting said medical instrument with an electrical source for cauterization through said tube.

17. The method of claim 12, wherein said assisting said medical instrument with said tube comprises:
assisting said medical instrument with a light source through said tube.

18. The method of claim 12, wherein said assisting said medical instrument with said tube comprises:
assisting said medical instrument with a plurality of tubes.

19. The method of claim 12, comprising:
disposing said tube along a length of said protective sheath.

20. The method of claim 12, comprising:
disposing said tube between an outer surface and an inner surface of said first portion.
